(19) United States
(12) Patent Application Publication
(10) Pub. No.: US 2013/0261723 A1
(43) Pub. Date: Oct. 3, 2013

(54) Treatment Of Diabetic Patients With A Drug Eluting Stent And A Drug Coated Balloon

(71) Applicants: John J. Stankus, Campbell, CA (US); Syed F.A. Hossainy, Hayward, CA (US); Daniel L. Cox, Palo Alto, CA (US); Paul M. Consigny, San Jose, CA (US)

(72) Inventors: John J. Stankus, Campbell, CA (US); Syed F.A. Hossainy, Hayward, CA (US); Daniel L. Cox, Palo Alto, CA (US); Paul M. Consigny, San Jose, CA (US)

(73) Assignee: Abbott Cardiovascular Systems Inc., Santa Clara, CA (US)

(21) Appl. No.: 13/436,662

(22) Filed: Mar. 30, 2012

(51) Int. Cl. A61F 2/82; A61F 2/84; A61M 25/10

(52) U.S. Cl. 623/1.11; 623/1.42

(57) ABSTRACT

Embodiments of the present invention include methods for the treatment, prevention, or amelioration of vascular disease and/or disorder in diabetic patients. The methods include implantation of a stent including a drug, and the use of a drug coated balloon. The DES may be a DES having a metal body and a coating including the drug, or a bioabsorbable stent with drug in the body of the stent, in a coating on the stent, or both in the body of the stent and in a coating on the stent.

### BACKGROUND OF THE INVENTION

1. Field of the Invention

This invention relates to methods of treating vascular disease and disorders in diabetic and pre-diabetic patients.

2. Description of the State of the Art

Until the mid-1980s, the accepted treatment for coronary atherosclerosis, i.e., narrowing of the coronary artery(ies) was coronary by-pass surgery. While being quite effective and having evolved to a relatively high degree of safety for such an invasive procedure, by-pass surgery still involves potentially serious complications and in the best of cases an extended recovery period.

With the advent of percutaneous transluminal coronary angioplasty (PTCA) in 1977, the scene changed dramatically. Using catheter techniques originally developed for heart exploration, inflatable balloons were employed to re-open (dilate) occluded regions in arteries. The procedure was relatively non-invasive, took a very short time compared to by-pass surgery and the recovery time was minimal. However, PTCA brought with it other problems such as vasospasm and elastic recoil of the stretched arterial wall which could undo much of what was accomplished and, in addition, created a new problem, restenosis, the re-clogging of the treated artery due to neointimal hyperplasia, that is, abnormal regrowth of the inner lining of the vessel after treatment with PTCA.

The next improvement, advanced in the mid-1980s, was the use of a stent to maintain the luminal diameter after PTCA. This for all intents and purposes put an end to vasospasm and elastic recoil but did not entirely resolve the issue of restenosis. That is, prior to the introduction of stents, restenosis occurred in from about 30 to 50% of patients undergoing PTCA. Stenting reduced this to about 15 to 20%, a substantial improvement but still more restenosis than desirable. For diabetic patients, however, after stenting, the incidence of restenosis and major cardiac events were significantly higher than for non-diabetics patients.

In 2003, drug eluting stents or DESs were introduced. The drugs employed with the DES are cytostatic compounds that curtailed the proliferation of cells that resulted in restenosis. The occurrence of restenosis has been reduced to about 5 to 7%, a very improved figure. However, based upon the studies to date, the rate of restenosis with DES remains higher for diabetic patients than non-diabetic patients. Thus, there is a need for improved methods for treating vascular diseases and disorders, more particularly in diabetic patients.

### SUMMARY OF THE INVENTION

The present invention is directed to methods of treating vascular diseases and disorders in diabetic and pre-diabetic patients. The methods include the use of a drug coated balloon and the implantation of a stent.

More specifically, various embodiments of the present invention encompass methods of treating, preventing, or ameliorating a vascular disease and/or disorder in a diabetic or pre-diabetic patient. The methods include delivering a balloon with a coating comprising a first drug to a vascular region in a patient, deploying the drug coated balloon (DCB) at the site of the vascular region to deliver the first drug, delivering a stent comprising a second drug to the vascular region of the patient, and deploying the stent comprising the second drug at the vascular region to deliver the second drug. The second drug may be the same as or different from the first drug. The patient is determined to have diabetes or to have a pre-diabetic condition and the patient is in need of treating, preventing, or ameliorating a vascular disease and/or disorder. Each of the first and the second drugs may be independently selected from the group consisting of anti-inflammatories, thiazolidinediones, antiproliferatives, and combinations thereof.

The antiproliferatives are paclitaxel, docetaxel, methotrexate, azathioprine, vincristine, vinblastine, fluorouracil, doxorubicin hydrochloride, mitomycin, rapamycin (sirolimus), Biolimus A9, deforolimus, AP23572, tacrolimus, temsirolimus, pimecrolimus, novolimus, zotarolimus (ABT-578), everolimus, 40-O-(3-hydroxypropyl)rapamycin, 40-O-[2-(2-hydroxy)ethoxy]ethyl-rapamycin, 40-O-(2-hydroxy)ethyl-rapamycin (everolimus), 40-O-tetrazolylrapamycin, 40-epi-(N1-tetrazolyl)-rapamycin, compound NVP-BEZ235 (an mTOR inhibitor and a PI3K inhibitor), and the following PI3K inhibitors LY294002 (available from Calbiochem), XL765 and XL147 (Exelixis/Sanofi-Aventis), GDC-0941 (Genentech/Roche), BKM120 (Novartis), BEZ235 (Novartis), AMG319 (Amgen), and CAL101 (aka GS1101)(Calistoga Pharmaceuticals/Gilead), and combinations thereof.

The anti-inflammatories are clobetasol, clobetasol propionate, clobetasone butyrate, dexamethasone, dexamethasone dipropionate, dexamethasone acetate, dexmethasone phosphate, momentasone, cortisone, cortisone acetate, hydrocortisone, prednisone, prednisone acetate, betamethasone, betamethasone acetate, and combinations thereof.

The thiazolidinediones are pioglitazone, rosiglitazone, troglitazone, netoglitazone, ciglitazone, rivoglitazone, and combinations thereof.

### BRIEF DESCRIPTION OF THE DRAWINGS

FIG. 1 is an elevational view, partially in section, of a typical stent mounted on a delivery catheter and disposed within a damaged artery.

FIG. 2 is an elevational view, partially in section, similar to that shown in FIG. 1 wherein the stent is expanded within a damaged artery.

FIG. 3 is an elevational view, partially in section, depicting the expanded stent within the artery after withdrawal of the delivery catheter.

FIG. 4 is a schematic depiction of a typical balloon of a catheter balloon where the balloon has been inflated within a lumen.

### DETAILED DESCRIPTION OF THE INVENTION

Use of the term “herein” encompasses the specification, the abstract, and the claims of the present application.

Use of the singular herein includes the plural and vice versa unless expressly stated to be otherwise, or obvious from the context that such is not intended. That is, “a” and “the” refer to one or more of whatever the word modifies. For example, “a drug” includes one drug, two drugs, etc. Likewise, “the polymer” may refer to one, two or more polymers, and “the anti-inflammatory” may mean one anti-inflammatory or a plurality of anti-inflammatories. By the same token, words such as, without limitation, “polymers” and “anti-inflammatories” would refer to one polymer or anti-inflammatory as well as to a plurality of polymers or anti-inflammatories unless, again, it is expressly stated or obvious from the context that such is not intended.

As used herein, unless specifically defined otherwise, any words of approximation such as without limitation, “about,” “essentially,” “substantially,” and the like mean that the element so modified need not be exactly what is described but can vary from the description. The extent to which the description may vary will depend on how great a change can be instituted and have one of ordinary skill in the art recognize the modified version as still having the properties, characteristics and capabilities of the unmodified word or phrase. In general, but with the preceding discussion in mind, a numerical value herein that is modified by a word of approximation may vary from the stated value by ±15%, unless expressly stated otherwise.

As used herein, any ranges presented are inclusive of the end-points. For example, “a duration of time between 10 and 75 minutes” or “a duration of time from 10 to 75 minutes” includes 10 minutes and 75 minutes, as well as any specific duration of time in between 10 minutes and 75 minutes.

As used herein, a “polymer” refers to a molecule comprised of repeating “constitutional units.” The constitutional units derive from the reaction of monomers. The constitutional units themselves can be the product of the reactions of other compounds. As a non-limiting example, ethylene (CH₂═CH₂) is a monomer that can be polymerized to form polyethylene, CH₃CH₂(CH₂CH₂)ₙCH₂CH₃ (where n is an integer), wherein the constitutional unit is —CH₂CH₂—, ethylene having lost the double bond as the result of the polymerization reaction. A polymer may be derived from the polymerization of two or more different monomers and therefore may comprise two or more different constitutional units. Such polymers are referred to as “copolymers.” “Terpolymers” are a subset of “copolymers” in which there are three different constitutional units. Those skilled in the art, given a particular polymer, will readily recognize the constitutional units of that polymer and will readily recognize the structure of the monomer from which the constitutional units derive. Polymers may be straight chain, branched chain, star-like or dendritic. One polymer may be attached (grafted) onto another polymer. The constitutional units of polymers may be randomly disposed along the polymer chain, may be present as discrete blocks, may be so disposed as to form gradients of concentration along the polymer chain, or a combination thereof. Polymers may be cross-linked to form a network.

As used herein, a polymer has a chain length of 50 constitutional units or more, and those compounds with a chain length of fewer than 50 constitutional units are referred to as “oligomers.”

As used herein, the terms “biodegradable,” “bioerodable,” “bioabsorbable,” “degraded,” “eroded,” “absorbed,” and “dissolved,” are used interchangeably, and refer to a substance that is capable of being completely or substantially, degraded, dissolved, eroded, or any combination thereof, over time when exposed to physiological conditions (pH, temperature, enzymes and the like), and can be gradually eliminated by the body, or that can be degraded into fragments that can pass through the kidneys. Conversely, “biostable” refers to a substance that is not biodegradable, etc.

As used herein, a material that is described as a layer, a film, or a coating “disposed over” a substrate refers to deposition of the material directly or indirectly over at least a portion of the surface of that substrate. “Directly deposited” means that the material is applied directly to the surface of the substrate. “Indirectly deposited” means that the material is applied to an intervening layer that has been deposited directly or indirectly over the substrate. The terms “layer,” “film,” and “coating” are used interchangeably herein. A coating may have multiple layers, and each layer may be applied by multiple applications of coating material. A single layer may be formed by application of more than one coating material or coating solution. Layers typically differ from each other in the type of materials, the ratio of materials, or both the type of and the ratio of materials applied to form the layer. Materials may migrate from one layer to another layer during the coating application process, after the coating has been formed or both during the coating application process and after the coating has been formed.

As used herein, a “drug” refers to a substance that, when administered in a therapeutically effective amount to a patient suffering from a disease, disorder, or condition, has a therapeutic beneficial effect on the health and well-being of the patient. A therapeutic beneficial effect on the health and well-being of a patient includes, but is not limited to: (1) curing the disease, disorder, or condition; (2) slowing the progress of the disease, disorder, or condition; (3) causing the disease, disorder, or condition to retrogress or to be in remission; or, (4) alleviating, ameliorating or both alleviating and ameliorating one or more symptoms of the disease, disorder, or condition.

As used herein, a “drug” also includes any substance that when administered to a patient, known or suspected of being particularly susceptible to a disease, in a prophylactically effective amount, has a prophylactic beneficial effect on the health and well-being of the patient. A prophylactic beneficial effect on the health and well-being of a patient includes, but is not limited to: (1) preventing or delaying on-set of the disease, disorder, or condition in the first place; (2) maintaining a disease, disorder, or condition at a retrogressed level once such level has been achieved by a therapeutically effective amount of a substance, which may be the same as or different from the substance used in a prophylactically effective amount; or, (3) preventing or delaying recurrence of the disease, disorder, or condition after a course of treatment with a therapeutically effective amount of a substance, which may be the same as or different from the substance used in a prophylactically effective amount, has concluded.

As used herein, “drug” also refers to pharmaceutically acceptable, pharmacologically active derivatives of those drugs specifically mentioned herein, including, but not limited to, salts, esters, amides, hydrates, solvates, and the like.

As used herein, the phrase “drug is X” also refers to pharmaceutically acceptable, pharmacologically active derivatives of the drug X, such as, but not limited to, salts, esters, amides, hydrates, solvates, and the like. As a non-limiting an example, “the drug is dexamethasone” would also encompass dexamethasone acetate.

As used herein, a “cardiovascular disease” is a disease, condition, or disorder that impacts the heart, circulatory system, or both the heart and the circulatory system. The circulatory system includes the cardiovascular system, and the lymphatic system. The lymphatic system distributes lymph. The cardiovascular system is a system of blood vessels, primarily arteries and veins, which transport blood to and from the heart, brain and peripheral organs such as, without limitation, the arms, legs, kidneys and liver. The coronary artery system supplies blood to the heart. The carotid artery system supplies blood to the brain. The peripheral vascular system carries blood to (via arteries) and from (via veins) the peripheral organs such as, without limitation, the hands, legs, kidneys and liver. The coronary artery system, carotid artery system, and the peripheral vascular system which includes the peripheral artery system are sub-systems of the cardiovascular system.

As used herein, a “vascular disease” generally refers to a disease, condition, or disorder that impacts the circulatory system. In particular “vascular disease” includes a disease, disorder, or condition of the coronary system, the carotid system and the peripheral vascular system.

“Vascular diseases” are a subset of “cardiovascular diseases.”

Examples of cardiovascular diseases include diseases of the heart which include, but are not limited to, heart valve disease, arrhythmia, heart failure, and congenital heart disease, and vascular diseases which include, but are not limited to atherosclerosis, thrombosis, restenosis, hemorrhage, vascular dissection or perforation, vulnerable plaque, chronic total occlusion, claudication, anastomotic proliferation for vein and artificial grafts, peripheral artery disease, carotid artery disease, coronary artery disease, anuerysm, renal (kidney) artery disease, raynaud's syndrome, buerger's disease, peripheral venous disease, varicose veins, blood clots in the veins, blood clotting disorders, and lymphdema.

As used herein, an “implantable medical device” refers to any type of appliance that is totally or partly introduced, surgically or medically, into a patient's body or by medical intervention into a natural orifice, and which is intended to remain there after the procedure. The duration of implantation may be essentially permanent, i.e., intended to remain in place for the remaining lifespan of the patient; until the device biodegrades; or until it is physically removed. Examples of implantable medical devices include, without limitation, vascular grafts, self-expandable stents, balloon-expandable stents, and stent-grafts.

With respect to an implantable medical device, the “outer surface” is meant any surface however spatially oriented that is in contact with bodily tissue or fluids.

With respect to an implantable medical device, a “device body” refers to an implantable medical device in a fully formed utilitarian state with an outer surface to which no coating or layer of material different from that of which the device itself is manufactured has been applied.

One type of implantable medical device is a stent. Stents are implantable medical devices that are generally cylindrically shaped, and function to hold open, and sometimes expand, a segment of a blood vessel or other lumen or vessel in a patient's body when the vessel is narrowed or closed due to diseases or disorders including, without limitation, coronary artery disease, carotid artery disease and peripheral arterial disease. A stent can be used in, without limitation, neuro, carotid, coronary, pulmonary, renal, biliary, iliac, femoral and popliteal, as well as other peripheral vasculatures, as well as other bodily lumens. A stent can be used in the treatment or prevention of vascular disorders, as well as other disorders. For a stent, the “outer surface” includes the luminal surface which faces the lumen interior, the abluminal surface which faces the lumen wall, and sidewall surfaces, if present, which connect the abluminal and luminal surfaces.

Another category of medical devices are insertable medical devices. “Insertable medical devices” include any type of appliance that is totally or partly introduced, surgically or medically, into a patient's body or by medical intervention into a natural orifice, but the device does not remain in the patient's body after the procedure.

A “catheter” is a thin, flexible tube for insertion into a natural body cavity, duct, or vessel, and may be used to introduce or remove fluid, to distend the vessel, or to hold open the vessel or cavity.

A “vascular catheter” is an example of an insertable medical device. A vascular catheter is a thin, flexible tube with a manipulating means at one end, which remains outside the patient's body, and an operative device at or near the other end, which is inserted into the patient's artery or vein. The catheter may be used for the introduction of fluids, often containing drugs, to the target site. The catheter may be used to deliver a stent to the target site, or may be used to deliver a balloon used in angioplasty. The catheter may perform multiple functions.

As used herein, a “balloon” comprises a relatively thin, flexible material, forming a tubular membrane, and is usually associated with a vascular catheter. When positioned at a particular location in a patient's vessel can be expanded or inflated to an outside diameter that is essentially the same as the inside or luminal diameter of the vessel in which it is placed. Balloons may be inflated, without limitation, using a liquid medium such as water or normal saline solution (where saline means including salt, typically sodium chloride), that is, saline that is essentially isotonic with blood.

A “balloon catheter” refers to a medical device which is a system of a catheter with a balloon at the end of the catheter.

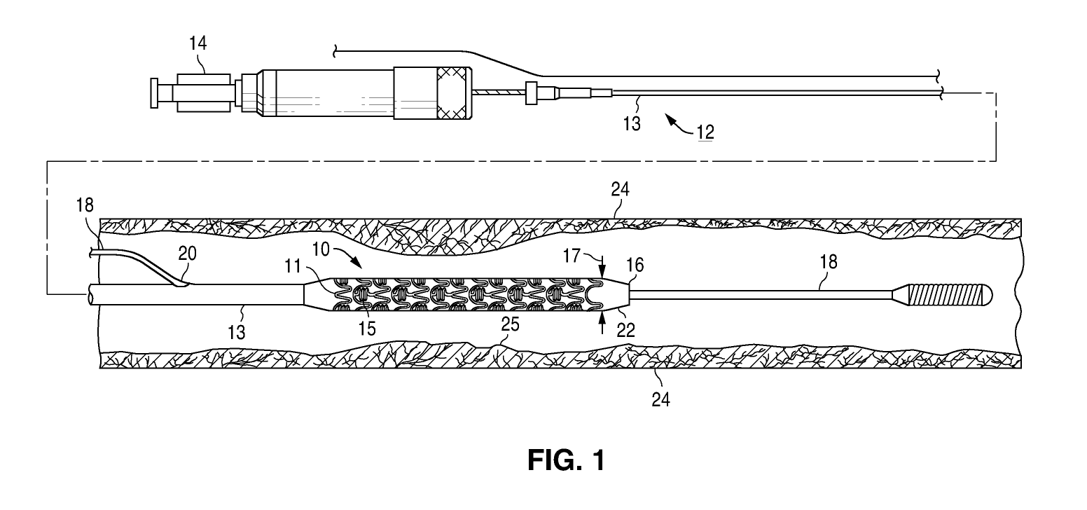

A typical implantation of a stent is described in the following paragraphs. FIG. 1 generally depicts a stent 10, mounted on a catheter assembly 12 which is used to deliver the stent 10 and implant it in a body lumen, such as a blood vessel 24. The non-limiting example of a stent 10 that is shown in FIG. 1 comprises a plurality of radially expandable cylindrical rings 11 disposed generally coaxially and interconnected by undulating links 15 disposed between adjacent cylindrical rings 11. The combination of cylindrical rings 11 and links 15 form the stent body, that is the device body of the stent (also referred to as the scaffolding), which supports the vessel once deployed. The catheter assembly 12 includes a catheter shaft 13 which has two ends, a first end 14 and a second end 16. The catheter assembly 12 is configured to advance through the patient's vascular system by advancing over a guide wire by any of the well known methods, including a rapid exchange catheter system, such as the one shown in FIG. 1. Another well known method for stent delivery is an over the wire system.

Catheter assembly 12 as depicted in FIG. 1 is of the well-known rapid exchange type which includes an RX port 20 where the guide wire 18 will exit the catheter from a lumen, which is a passageway or cavity, in the shaft 13. The distal end of the guide wire 18 exits the catheter second end 16 so that the catheter advances along the guide wire on a section of the catheter between the RX port 20 and the catheter second end 16. If the stent is of the balloon-expandable type, the stent is mounted on a balloon 22 and is crimped tightly thereon so that the stent 10 and balloon 22 present a low profile diameter for delivery through the arteries. Alternatively, a self-expanding stent configuration as is well known in the art may be used.

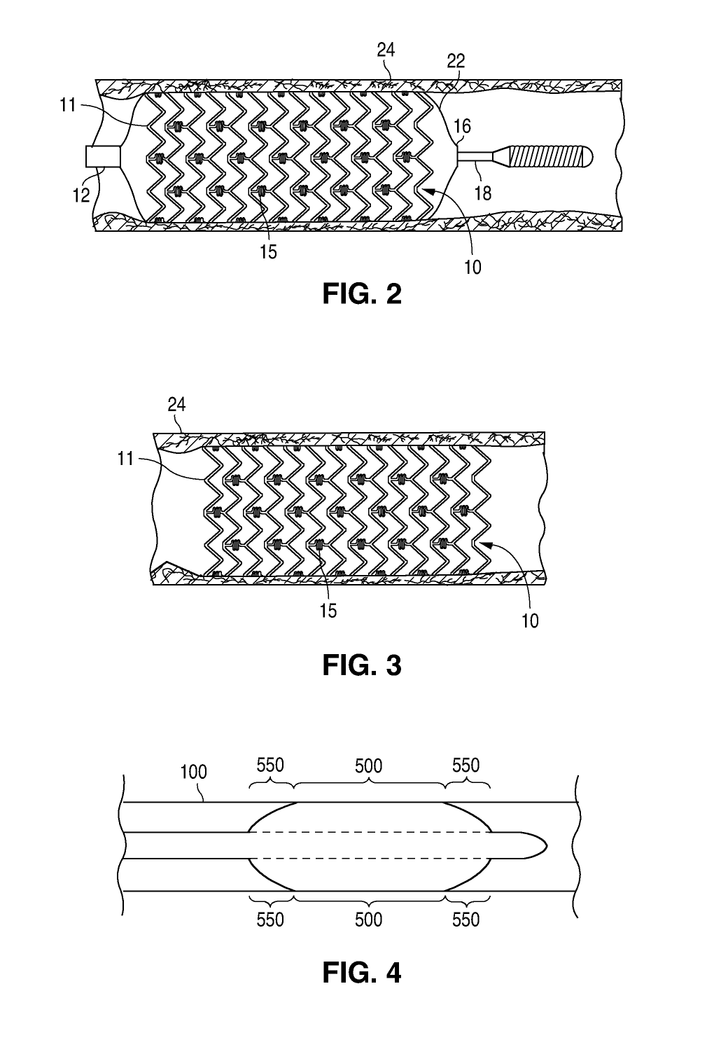

As shown in FIG. 1, a partial cross-section of an artery 24 is shown with a small amount of plaque 25 that has been previously treated by a repair procedure. A stent 10 may be used to repair a diseased or damaged arterial wall which may include the plaque 25 as shown in FIG. 1, or a dissection, or a flap which are commonly found in the coronary arteries, carotid arteries, peripheral arteries and other vessels. In a typical procedure to implant stent 10, the guide wire 18 is advanced through the patient's vascular system by well known methods so that the distal end of the guide wire is advanced past the plaque or diseased area 25. The introduction of the stent into the body and transport to a region that is to be treated is referred to herein as “delivery.” Once the stent 10 has been delivered to the region to be treated, the stent delivery catheter assembly 12 is advanced over the guide wire so that the stent 10 is positioned in the target area. The balloon 22 is inflated by well known means so that it expands radially outwardly and in turn expands the stent 10 radially outwardly until the stent is apposed to the vessel wall. The radial expansion of the stent, by a balloon or otherwise, until the stent is apposed to the vessel wall is referred to herein as “deployment” of the stent. The balloon 22 is then deflated and the catheter withdrawn from the patient's vascular system. The guide wire 18 typically is left in the lumen for post-stent implantation procedures, if any, and subsequently is withdrawn from the patient's vascular system. A lumen in the catheter shaft 13 may be used to deliver fluids, potentially including a drug, to the site, such as the site of plaque 25. As depicted in FIGS. 2 and 3, the balloon 22 is fully inflated with the stent 10 expanded and pressed against the vessel wall, and in FIG. 3, the implanted stent 10 remains in the vessel after the balloon 22 has been deflated and the catheter assembly 12 and guide wire 18 have been withdrawn from the patient. As used herein, “implantation” of a stent refers to the delivery and deployment of the stent.

As obvious from the preceding discussion, a balloon, a catheter, and a stent perform different functions. A stent is typically crimped to a smaller diameter for delivery, and then the stent subsequently expands if self-expanding, or is expanded by a balloon or other device, to a large diameter. The expanded stent is capable of supporting a bodily lumen for an extended period of time. In contrast, a balloon has a wall thickness that is so thin that the tubular membrane cannot support a load at a given diameter unless inflated with a fluid. Furthermore, a balloon is a transitory device that is inserted in the patient's body for only a limited time for the purpose of performing a specific procedure or function. Dilatation balloons used to expand a vessel wall, and optionally open an occluded vessel, are not implanted, but are removed from the body at the end of the procedure. Catheters have a shaft which is similar to a stent in that most stents and catheter shafts are tubular or cylindrical in shape. However, a catheter shaft is not designed to be radially expandable. In addition, a vascular catheter has a much larger (a factor of 10 or greater) length to diameter ratio than a stent.

As discussed previously, the use of stents has reduced the incidence of restenosis, but to a lower extent in diabetic patients. For example, one study found that after a percutaneous cardiac intervention followed by the implantation of a bare metal stent (a stent having a metal device body with no coating over the outer surface), the rate of restenosis was 30% for diabetic patients compared to 20% for non-diabetic patients. Another study involving implantation of a DES, found a rate of 14.6% restenosis in non-diabetics, but 20.9% for diabetic patients. In addition, diabetic patients are more likely to experience major adverse cardiac events (MACE) after PTCA with stenting. In general, diabetics are more than twice as likely as non-diabetics to have a heart attack or stroke, and 2 out of 3 diabetics die from cardiovascular disease (American Diabetes Association). Hyperglycemia, independent of whether or not a person has been diagnosed with diabetes, is a risk-factor for cardiovascular events.

Diabetic patients are those individuals suffering from diabetes mellitus, often referred to as just “diabetes,” a group of metabolic diseases. Diabetes may be type 1, previously referred to as juvenile diabetes, in which an individual is unable to produce insulin. Type 1 diabetes may also be called insulin dependent diabetes. Type 2 diabetes results from an insulin level which is too low, or an inability to utilize insulin, referred to as “insulin resistance.” As used herein, a person may be diagnosed as diabetic if at least one of the following applies:

(1) fasting plasma glucose level is greater than or equal to 7.6 mmol/L (126 mg/dL);

(2) plasma glucose level is greater than or equal to 11.0 mmol/L (200 mg/dL) 2 hours after a 75 gram oral glucose load (standard glucose tolerance test);

(3) symptoms of hyperglycemia (described below), and a “casual” plasma glucose of greater than or equal to 11.1 mmol/L;

(4) glycated hemoglobin (a.k.a. hemoglobin A1C or HbA1C) of greater than or equal to 6.5%.

In general, the measurements should be repeated on more than one day for a definitive diagnosis of diabetes. Hyperglycemia is a condition of high plasma glucose. Symptoms of hyperglycemia include increased thirst and urination, increased hunger, blurred vision, feelings of weakness, weight loss, and dry mouth. Those people in which at least one of the following apply, (1) a fasting blood glucose that is 5.6 to 6.9 mmol/liter (100 to 125 mg/dL), and (2) a glucose tolerance test plasma glucose level of 7.8 to 11.1 mmol/liter (140 to 200 mg/dL), are classified as “pre-diabetic.”

As used herein, a “diabetic patient” is an individual (animal, including human) who has been diagnosed as having diabetes, either type 1 or type 2, or an individual, although not diagnosed as diabetic, who would be diagnosed as a diabetic individual if that individual were to be evaluated. As an example, for a human, if the plasma glucose or HbA1C, if measured, would fall within the range described above that is classified as diabetic, that individual would be classified as a “diabetic patient,” even if not formally diagnosed. Different criteria may apply to individuals of different species. The methods of the present invention encompass treatment of those individuals classified as diabetic under current clinical criteria, as well as those who classify as diabetic under any criteria as revised or developed in the future. Those referred to as “pre-diabetic” individuals would be determined analogously.

It is believed that there are a number of reasons that diabetics exhibit higher rates of cardiovascular disease. Diabetics suffer from endothelial dysfunction making diabetics more prone to vascular lesions. The high blood glucose levels may damage heart muscle, and increase oxidative stress. Many diabetic patients have “atherogenic dyslipidemia,” or an abnormal lipid profile in the blood. This abnormal lipid profile is characterized by elevated triglycerides, and low levels of high density lipoprotein (HDL) cholesterol. Even if the low density lipoprotein (LDL) cholesterol, also referred to as “bad cholesterol,” is at a normal level, the actual LDL particles are often abnormal, such as by being smaller or denser, or both smaller and denser, and as a result, more likely to lead to atherosclerosis. Inflammation also plays a role in the development of diabetes, and plasma levels of inflammatory molecules and adhesion molecules are elevated in diabetic patients. In fact, some have referred to type II diabetes as a “chronic inflammatory disease.” At least one study has found a correlation between blood markers of inflammation and the propensity to become diabetic, but the correlation was not applicable to African Americans and smokers. Animal models have shown that T cells and macrophages, both involved in immune response, are involved in the development of diabetes or insulin resistance.

Vascular diseases may also involve inflammatory processes. It is believed that the atherosclerosis plaque formation initiates with the stimulation of VCAM-1 (vascular cell adhesion molecule-1) by endothelial cells in the wall of the artery. “Atherosclerosis” refers to the depositing of fatty substances, cholesterol, cellular waste products, calcium and fibrin on the inner lining, or intima, of an artery. Smooth muscle cell proliferation and lipid accumulation accompany the deposition process. Stimulation of VCAM-1 is thought to occur by oxidized lipids. Another pathway for stimulation of VCAM-1 involves nuclear factor-κB. VCAM-1 may also be stimulated by proinflammatory cytokines. Cytokines are small cell-signaling proteins. An example of a proinflammatory cytokine that may stimulate VCAM-1 is IL-1β, interluenkin-1β. VCAM-1 may also be stimulated by a substance called TNF-α, tumor necrosis factor-α. Specifically, the stimulation of VCAM-1 results in the adhesion of white blood cells, including immune modulated white blood cells. The white blood cells within the vessel wall eventually become macrophages, which are involved in immune response by engulfing and digesting cellular debris and pathogens. In the development of atherosclerosis, the macrophages engulf modified lipoproteins in the blood, particularly LDL. In a cascade effect, the macrophages also produce growth factors and cytokines, which are proinflammatory, thus attracting more white blood cells. Eventually the macrophages become the foam cells seen in atherosclerotic plaque.

Atherosclerotic plaque, also called fibrous (atheromatous) plaques and atherosclerotic lesions, result from the accumulation of substances on the intima and reduce the lumen of the artery, and create a stenosis. When the stenosis becomes severe enough, the blood supply to the organ supplied by the particular artery is depleted resulting in strokes, if the afflicted artery is a carotid artery, heart attack if the artery is a coronary artery, or loss of organ function if the artery is peripheral.

Stenting and PTCA can injure the vessel wall, such as by causing endothelial denudation, and the injury may cause inflammation. Inflammation may result in changes to smooth muscle cells with over-proliferation of muscle cells and migration of these cells into the intima. It is the overgrowth of cells that may lead to restenosis, that is the re-growth of the previously repaired stenosis. Thus, the vascular injury caused by stenting may eventually lead to restenosis.

Because diabetics suffer from endothelial dysfunction and inflammation, diabetics may be particularly susceptible to restenosis. It is interesting to note that the risk factors for cardiovascular disease and diabetes significantly overlap.

At least one clinical study involving implantation of DES showed a correlation between one inflammatory marker, high sensitive C reactive protein (CRP), in the blood of diabetic patients and rates of restenosis. Another study, which involved implantation of bare metal stents, showed no correlation with a large number of inflammatory markers and rates of restenosis. However, both of the above referenced studies concluded that control of diabetes, as determined by HbA1C, plasma glucose, or both, was a factor in the rate of restenosis. Moreover, the study utilizing bare metal stents found that the fasting blood glucose level at the time of stenting correlated with the rate of restenosis. Thus, treatment of underlying inflammation or diabetes may help reduce restenosis.

Embodiments of the present invention include methods for the treatment, prevention, or amelioration of vascular disease and disorders in diabetic and pre-diabetic patients. The methods include both use of a drug coated balloon including a first drug and implantation of a stent including a second drug in a vascular region of a patient (animal, including human) in need of treatment, prevention, or amelioration of a vascular disease and/or disorder. The patient is determined to have a diabetic or pre-diabetic condition. It is believed that restenosis or another vascular disease may be prevented, treated, or ameliorated by the administration of a first drug via the local delivery of a drug coated balloon. The second drug and the first drug may be an anti-proliferative, an anti-inflammatory, either steroidal or non-steroidal, a thiazolidinedione (also referred to as a glitazone), or a combination thereof.

Vascular regions or sites that may benefit from treatment include, but are not limited to, vascular lesions, atherosclerotic lesions, site of vulnerable plaque(s), and the site of a peripheral arterial disease. An atherosclerotic lesion refers to a deposit of fatty substances, cholesterol, cellular waste products, calcium, fibrin, or a combination thereof on the inner lining or intima of an artery. A peripheral artery disease site may be a similar lesion in a peripheral artery that is also caused by the buildup of fatty deposits on the lining or intima of the artery walls. Examples of vascular lesions include, without limitation, saphenous vein graft lesions, restenotic lesions, bifurcation lesions, ostial lesions, left main lesions, chronic total occlusions and occlusions associated with AMI (Acute Myocardial Infarction), and STEMI (ST-segment Elevation Myocardial Infarction).

“Vulnerable plaque” refers to an atheromatous plaque that has the potential of causing a thrombotic event (formation of a clot within the vessel that blocks the vessel), and is usually characterized by a very thin wall separating it from the lumen of an artery. The thinness of the wall renders the plaque susceptible to rupture. The walls are formed from collagen which may be negatively impacted by inflammation as well as other substances present in the blood stream. When the plaque ruptures, the inner core of usually lipid-rich plaque is exposed to blood, with the potential of causing a fatal thrombotic event through adhesion and activation of platelets and plasma proteins to components of the exposed plaque.

A drug coated balloon (DCB) is a balloon catheter that has a coating of a drug or a coating containing a drug disposed over at least a portion of the outer surface of the balloon. When the balloon is inflated, and the balloon walls contact the vessel walls, the drug is released. The underlying structure of the balloon can be virtually any structural design and the balloon can be composed of any suitable material. Non-limiting examples of suitable balloon materials polyester, PEBAX® (polyether block amide block copolymers, Arkema), polyurethanes, poly(tetrafluoroethylene) (aka PTFE, and TEFLON®, DuPont Co., Wilmington, Del.), nylon, and DACRON® (DuPont Co.)

A drug coating disposed over all or a portion of the outer surface of the balloon may include only a drug, or the coating may include a drug in combination with an excipient, and/or another material, such as a polymer. The coating may be constructed of multiple layers; the coating may be applied as multiple layers, or both. Multiple layers may have different materials, different ratios of materials, or both in each layer. The coating that is deposited over at least a portion of the outer surface of the stent may have a thickness of less than or about equal to 250 μm, preferably less than or about equal to 150 μm, and even more preferably, less than or about equal to 75 μm. These dimensions may apply to an individual layer if more than one layer is deposited on the balloon, to the total of all layers, or to both an individual layer and to the total of all layers. The balloon can be in a folded or unfolded state during the coating application. Typically, a balloon is selectively coated on the cylindrical surface corresponding to the “working length” of the balloon of a balloon catheter, which is the surface area of the balloon that would contact the lumen wall. As shown in FIG. 4, the ends 550 of balloon 520 are not necessarily square so that the balloon curves down to join the catheter tube, but the major portion of balloon length 500, which comprises the interface in contact with the lumen 100, has substantially the same diameter. For a balloon having substantially a single diameter over its entire length, such as that illustrated in FIG. 4, the interface in contact with the lumen, 500, may also be referred to as the “working length” or “working area.”

Non-limiting examples of materials that may be used in a coating disposed over at least a portion of a balloon include excipients, and hydrophilic polymers and oligomers, such as, without limitation, polyvinylpyrrolidone (PVP), poly(carboxymethyl cellulose) (poly(CMC)), poly(ethylene glycol) (PEG), poly[N-(2-hydroxypropyl)methacrylamide] (poly(HPMA)), and poly(vinyl alcohol); natural polymers and oligomers such as chitosan, and sodium alginate; cellulose based materials such as, but not limited to, hydroxyethyl cellulose, hydroxypropyl cellulose, methyl cellulose, hydroxypropyl methyl cellulose, poly(carboxymethyl cellulose), and ethyl cellulose; sugars and carbohydrates such as, without limitation, dextran, dextrin, starch, dextrose, sucrose, mannitol, sorbitol, xylitol, and trehalose; amphiphilic polymers and oligomers such as, but not limited to, poly(carboxymethyl cellulose), poly(n-butyl methacrylate-phosphorylcholine) (PBMA-PC), poly(ester amide)-phosphorylcholine (PEA-PC), polylactide-phosphorylcholine (PLA-PC), polylactide-phosphorylcholine (PLA-PC), polyethylene glycol-poly(caprolactone)-di- or tri-block copolymers or oligomers (PEG-PCL), polyethylene glycol-polylactide di- or tri-block copolymers or oligomers (PEG-PLA), polyethylene glycol-poly(lactide-glycolide) di- or tri-block copolymers or oligomers (PEG-PLGA), PLURONIC® (poly(ethylene oxide)-b-poly(propylene oxide)-b-poly(ethylene oxide)) di- and tri-block copolymers, etc. The term “poloxamer” refers to tri-block copolymers with a central block of poly(propylene oxide) (PPO) and with a block of poly(ethylene oxide) (PEO) on each side where the PEO blocks are usually of the same length in terms of number of constitutional units. Poloxamers of types 124, 188, 237, 338, and 407 are specified by a monograph in the National Formulary. Some PLURONIC® polymers sold by BASF also meet one of the NF specifications for a type of poloxamer. Excipients that may also be used in the coating include, but are not limited to, contrast agents, penetration enhancers, plasticizers, and mucoadhesives. Some non-limiting examples of these excipients include polysorbates (an example of which is TWEEN™ 60), glycerol, Vitamin E TGPS, N-methyl-2-pyrrolidone (NMP), and dimethyl sulfoxide (DMSO). Vitamin E TPGS is also known as D-alpha tocopheryl polyethylene glycol 1000 succinate, and is a water soluble form of Vitamin E. A specification for Vitamin-E TPGS is listed in the United States National Formulary (NF). Polysorbates are a group of oleate esters of sorbitol and its' anhydrides condensed with polymers of ethylene oxide. Polysorbates are used as emulsifiers and surfactants in food, pharmaceuticals and cosmetics. Examples include polysorbate 20, polysorbate 60, and polysorbate 80, the specifications of which are all listed in the United States Pharmacopeia (USP).

With a DCB, the balloon walls contact the vessel walls when inflated, and the drug is released. Therefore, the drug may be released during the actual inflation or when it is in contact with the vessel wall. In practice, a therapeutic amount of the drug is delivered to the vessel wall while limiting or reducing systemic delivery. The dose may be delivered over a few seconds, a few minutes, or up to a few hours. Thus, within the first 3 hours after expansion, at least 10% of the drug loading (total content of drug, or the amount of drug per device), preferably at least 25% of the total drug loading, more preferably at least 50%, and even more preferably at least 75% of the total drug loading is released. In some embodiments, at least 25% of the drug loading, preferably 50% of the total drug loading, and more preferably 75% of the total drug loading is released within the first 5 minutes following expansion. In some embodiments, the DCB may release not less than 25% of the drug within the first 3 minutes following the initiation of expansion, or not less than 25% of the drug within the first 2 minutes following the initiation of expansion. For a DCB, unreleased drug is the drug remaining on the device if removed from the patient, all of the remainder having been released, and preferably a limited amount (not more than 50% of the total content) is released systemically. The amount of drug that may be released systemically may depend upon the specific pharmacokinetics and pharmacodynamics of the drug disposed on the DCB.

In the various embodiments of the present invention, the use of the DCB and the implantation of the stent in a diabetic or pre-diabetic patient may occur within a 3 month time frame, a 1 month time frame, a 1 week time frame, or a 48 hour time frame, and preferably in a 24 hour time frame, more preferably within a 12 hour time frame, and even more preferably within the same interventional procedure. The DCB may be inserted and deployed and the stent may be implanted within the same operation, which is during the time that the patient is in the operating room for the stent implantation, the DCB is also delivered and deployed to release drug from the DCB. The DCB may be used prior to dilatation of the vessel, such as with an angioplasty balloon to treat the vascular region, or post dilatation. The DCB may also perform both functions, dilatation of the vessel and delivery of the drug. The stent may be delivered and deployed after dilatation of the vessel, or during the same procedure as dilatation, that is the balloon that deploys the stent may also dilate the vessel. The stent may be crimped onto the DCB, and thus the DCB may be used to expand the stent as well as to deliver drug. In some embodiments, the DCB performs the functions of delivery of the first drug, expansion of the stent, and dilatation of the vessel. The DCB may be used after both dilatation of the vessel and implantation of the stent. In other words, the DCB may be expanded within the expanded vessel and implanted stent to deliver drug.

The DCB may be used, or the initiation of the expansion of the DCB may begin, within 30 to 90 minutes, within 5 to 75 minutes, within 10 to 45 minutes, within 5 to 30 minutes, within 0 to 20 minutes, or within 15 minutes prior to the insertion of the delivery device to deliver the stent into the patient, or after dilatation of the vessel has ceased (the dilatation balloon is deflated). The DCB may be administered at the site of the implantation within minutes, for example within 60 minutes, within 30 minutes, within 20 minutes, within 10 minutes, within 5 minutes, or within 2 minutes of the deployment of the stent at the site. If the DCB is used to deliver and deploy the stent, the DCB can administer the drug as the stent is being deployed.

The DCB may be expanded using a pressure in the range of about 4 atmospheres to about 20 atmospheres or more.

The stent comprising the second drug may be of any design. The stent may be a self-expanding stent, or a balloon expandable stent. The stent may be formed from a polymer, a metal, a metal alloy, a ceramic, a glass, or any combination thereof. The stent may be made from a biostable material, a biodegradable material, or any combination thereof.

Non-limiting examples of metals and metal alloys that may be used to form the device body of the stent or a portion of the stent include, but are not limited to, ELASTINITE® (Guidant Corp.), NITINOL® (Nitinol Devices and Components), stainless steel, tantalum, tantalum-based alloys, nickel-titanium alloy, platinum, platinum-based alloys such as, for example, platinum-iridium alloys, iridium, gold, magnesium, titanium, titanium-based alloys, zirconium-based alloys, alloys comprising cobalt and chromium (ELGILOY®, Elgiloy Specialty Metals, Inc.; MP35N and MP20N, SPS Technologies) or combinations thereof. The trade names “MP35N” and “MP20N” describe alloys of cobalt, nickel, chromium and molybdenum. The MP35N consists of 35% cobalt, 35% nickel, 20% chromium, and 10% molybdenum. The MP20N consists of 50% cobalt, 20% nickel, 20% chromium, and 10% molybdenum.

The device body of the stent may be formed from, the device body may comprise, and/or the device body may comprise at least 50% by volume, a biodegradable material, such as, but not limited to, a biodegradable polymer. Examples of preferred biodegradable polymers include, but are not limited to, biodegradable polyanhydrides, biodegradable poly(ether-esters), biodegradable polyesters such as poly(lactide), poly(lactide-co-glycolide), poly(lactide-co-caprolactone), and poly(glycolide-co-caprolactone).

As used herein, “lactide” encompasses L-lactide, D, L-lactide, D-lactide, meso-lactide, and any combination thereof, unless one type is specifically recited.

As used herein, the terms poly(D,L-lactide), poly(L-lactide), poly(D,L-lactide-co-glycolide), and poly(L-lactide-co-glycolide) are used interchangeably with the terms poly(D,L-lactic acid), poly(L-lactic acid), poly(D,L-lactic acid-co-glycolic acid), and poly(L-lactic acid-co-glycolic acid), respectively.

The stent comprising the second drug may be of any type of DES. Examples include, but are not limited to, stents with depots, holes, grooves, pores, indentations, or a combination thereof in the surface where the second drug is partially or completely contained within at least a portion of the depots, holes, grooves, pores, indentations or combinations thereof; porous or hollow stents where the second drug may be contained within the pores or within the hollow interior of the stent; and stents with a drug coating disposed over at least a portion of the outer surface. If the stent has a coating including a drug disposed over all or a portion the outer surface of the stent, the coating may include only a drug, or the coating may include a drug in combination with another material such as, without limitation, a polymer, a metal, a metal alloy, a ceramic, a glass, or any combination thereof. The coating may be constructed of multiple layers; the coating may be applied as multiple layers, or both. Multiple layers may differ in the materials in each layer, the ratio of materials in each layer, or both. The coating that is deposited over at least a portion of the outer surface of the stent may have a thickness of less than or about equal to 30 μm, less than or about equal to 20 μm, less than or about equal to 10 μm, less than or about equal to 5 μm, or less than or about equal to 3 μm. These dimensions may apply to an individual layer if more than one layer is deposited on the outer surface of the stent, to the total of all layers, or to both an individual layer and to the total of all layers. The coating may be selectively applied to only a portion of the outer surface, such as for example, without limitation, the abluminal surface. The stent can be in an expanded or unexpanded state during the coating application. Any methods of coating may be used such, but not limited to, the well-known methods in the art of dipping, spraying, brushing, etc. The stent may be a bioabsorbable stent where the second drug is included in the device body of the stent. As an example, the stent may have a bioabsorbable polymeric device body with the drug homogenously, or substantially homogeneously, dispersed within the device body of the stent.

Polymers that may be used, individually or in combination, to prepare coatings that are disposed over a stent, to form the device body, to form a material from which the device body is constructed, or to form a part of a stent include, but are not limited to, poly(N-acetylglucosamine) (Chitin), Chitosan, poly(3-hydroxyvalerate), poly(lactide-co-glycolide), poly(3-hydroxybutyrate), poly(4-hydroxybutyrate), poly(3-hydroxybutyrate-co-3-hydroxyvalerate), polyorthoesters, polyanhydrides, poly(glycolic acid), poly(glycolide), poly(lactide), poly(L-lactide-co-D,L-lactide), poly(caprolactone), poly(lactide-co-caprolactone), poly(glycolide-co-caprolactone), poly(trimethylene carbonate), poly(glycolic acid-co-trimethylene carbonate), co-poly(ether-esters) (e.g. PEO/PLA), polyphosphazenes, biomolecules (such as fibrin, fibrin glue, fibrinogen, cellulose, starch, collagen and hyaluronic acid, elastin and hyaluronic acid), polyurethanes, silicones, polyesters, polyolefins, polyisobutylene and ethylene-alphaolefin copolymers, acrylic polymers and copolymers, vinyl halide polymers and copolymers (such as polyvinyl chloride), polyvinyl ethers (such as polyvinyl methyl ether), polyvinylidene halides (such as polyvinylidene chloride), polyacrylonitrile, polyvinyl ketones, polyvinyl aromatics (such as polystyrene), polyvinyl esters (such as polyvinyl acetate), acrylonitrile-styrene copolymers, ABS resins, polyamides (such as Nylon 66 and polycaprolactam), polycarbonates including tyrosine-based polycarbonates, polyoxymethylenes, polyimides, polyethers, rayon, rayon-triacetate, cellulose, cellulose acetate, cellulose butyrate, cellulose acetate butyrate, cellophane, cellulose nitrate, cellulose propionate, cellulose ethers, and carboxymethyl cellulose. Additional representative examples of polymers include ethylene vinyl alcohol copolymer (commonly known by the generic name EVOH or by the trade name EVAL®), poly(butyl methacrylate), poly(vinylidene fluoride-co-hexafluoropropene) (e.g., SOLEF® 21508, available from Solvay Solexis PVDF, Thorofare, N.J.), polyvinylidene fluoride (an example is KYNAR®, available from Atofina Chemicals, Philadelphia, Pa.), ethylene-vinyl acetate copolymers, poly(vinyl acetate), styrene-isobutylene-styrene triblock copolymers, polyethylene glycol, poly(ester amide) polymers, polyacrylates, polymethacrylates, and phosphorylcholine substituted polymers such as, without limitation, polyesteramides. A specific non-limiting example of a phosphorylcholine substituted polymer is the phosphorylcholine-linked methacrylate polymer, 1036 (PC-1036™ from Biocompatibles Ltd, Farnham, Surrey, United Kingdom), poly(2-(methacryloyloxyethyl)-2′-(trimethylammoniumethyl)phosphate, inner salt)-co-(dodecyl methacrylate)-co-(2-hydroxypropylmethacrylate)-co-(3-Trimethoxysilyl)propylmethacrylate (23:47:25:5 mole %), which is a random copolymer of four components. The structure of PC-1036 is the following:

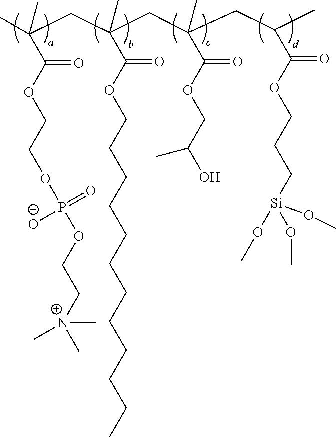

In the above illustration, a, b, c, and d stand for the stoichiometric (theoretical) ratios of each monomer. There are multiple monomers in the polymer. The above polymers may be used individually, or in any combination of two or more polymers in any proportion, and may be used with other materials.

A coating disposed over a stent, that is over at least a portion of a surface of the stent, or over at least a portion of a balloon may also include suspending agents, surfactants, lubricating agents, fillers, plasticizing agents, diluents, agents which act as active agent carriers or binders, anti-tack agents, anti-foaming agents, viscosity modifiers, anti-oxidants, stabilizers, and potentially residual levels of solvents.

Materials used in coating disposed over at least a portion of a balloon or a stent that are not expected to remain attached to the balloon or to a biostable stent until removed from the body are preferably biodegradable, and/or are of a sufficiently low molecular weight (not more than 40,000 Dalton) to pass through the kidneys.

The duration of release of the second drug, or the time at which about 80% of the second drug has been released, may be in the range of from about 1 to 3 days, to a year or more. In preferred embodiments the second drug is released over a time period of about 1 week to about 12 months, more preferably 2 weeks to 8 months, and even more preferably over a period of about 1 month to about 6 months.

The drug in a coating disposed over at least a portion of a stent or a balloon may be in the form of particles, where the particles may be of only a drug, substantially only a drug, a drug in combination with another material, including, but not limited to, a polymer, or a combination thereof.

The first drug and the second drug may be an anti-proliferative, an anti-inflammatory, a thiazolidinedione (a glitazone), or a combination thereof. The anti-proliferative may be a mitotic inihibitor such as a taxane. Non-limiting examples of taxanes include paclitaxel (TAXOL), and docetaxel (TAXOTERE®). The anti-proliferative may be an mTOR inhibitor. Non-limiting examples of mTOR inhibitors include rapamycin (sirolimus), Biolimus A9, deforolimus, AP23572 (Ariad Pharmaceuticals), tacrolimus, temsirolimus, pimecrolimus, novolimus, zotarolimus (ABT-578), 40-O-(2-hydroxy)ethyl-rapamycin (everolimus), 40-O-(3-hydroxypropyl), 40-O-[2-(2-hydroxy)ethoxy]ethyl-rapamycin, 40-O-tetrazolylrapamycin, and 40-epi-(N1-tetrazolyl)-rapamycin. The anti-proliferative may be a phosphatidylinositol 3-kinase inhibitor (PI3K inhibitor). Non-limiting examples of PI3K inhibitors include LY294002 (available from Calbiochem), XL765 and XL147 (Exelixis/Sanofi-Aventis), GDC-0941 (Genentech/Roche), BKM120 (Novartis), BEZ235 (Novartis), AMG319 (Amgen), and CAL101 (aka GS1101)(Calistoga Pharmaceuticals/Gilead). The compound NVP-BEZ235 is both an mTOR inhibitor and a PI3K inhibitor. Other anti-proliferative drugs include, without limitation, suramin and cytostatic drugs, such as methotrexate, azathioprine, vincristine, vinblastine, fluorouracil, doxorubicin hydrochloride, mitomycin, and actinomycins.

Non-limiting examples of anti-inflammatory drugs including both steroidal and non-steroidal (NSAID) anti-inflammatories, include clobetasol, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, deflazacort, desonide, desoximetasone, dexamethasone, dexamethasone dipropionate, dexamethasone acetate, dexmethasone phosphate, momentasone, cortisone, cortisone acetate, hydrocortisone, prednisone, prednisone acetate, betamethasone, betamethasone acetate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, methylprednisolone suleptanate, morniflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, zomepirac sodium, tacrolimus and pimecrolimus.

Examples of steroidal anti-inflammatory drugs include, without limitation, 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, any of their derivatives, and combinations thereof.

Examples of nonsteroidal anti-inflammatory drugs include, without limitation, COX-1 and COX nonspecific inhibitors (e.g., salicylic acid derivatives, aspirin, sodium salicylate, choline magnesium trisalicylate, salsalate, diflunisal, sulfasalazine and olsalazine; para-aminophenol derivatives such as acetaminophen; indole and indene acetic acids such as indomethacin and sulindac; heteroaryl acetic acids such as tolmetin, dicofenac and ketorolac; arylpropionic acids such as ibuprofen, naproxen, flurbiprofen, ketoprofen, fenoprofen and oxaprozin), and selective COX-2 inhibitors (e.g., diaryl-substituted furanones such as rofecoxib; diaryl-substituted pyrazoles such as celecoxib; indole acetic acids such as etodolac and sulfonanilides such as nimesulide), and combinations thereof.

Non-limiting examples of thiazolidinediones are repaglinide and natalinide.

The drugs may be used individually, or in combination with one or more other drugs.

The dose of the first drug may be in the range of about 1 to about 1000 μg/cm², and the dose of the second drug may range from about 1 to about 1000 μg/cm², if present in a coating disposed over at least a portion of the stent, and from about 0.1 to about 1000 μg/cm³, if present in the device body of the stent. If the second drug is an mTOR inhibitor, the drug dose may range from 1-1000 μg/cm², 10-600 μg/cm², preferably from 20 to 400 μg/cm², and more preferably from 30 to 300 μg/cm², if present in a coating disposed over at least a portion of the stent, and from 0.1-1000 μg/cm³, 0.2-600 μg/cm³, preferably from 0.3 to 400 μg/cm³, and more preferably from 0.5 to 300 μg/cm³, if present in the device body of the stent. In some embodiments, the DCB may have a drug loading between 10 to 1000 μg/cm², and preferably 50 to 750 μg/cm². In still other embodiments, the DCB drug loading may be in the range from 100 to 600 μg/cm², 150 to 600 μg/cm², 250 to 550 μg/cm², or 300 to 500 μg/cm². In some embodiments, the dose of the second drug may range from 0.1 to 100 μg per cm of stent length, preferably 1 to 75 μg/cm, and more preferably, 5 to 50 μg/cm, whether the second drug is included in the body of the stent, in a coating disposed over at least a portion of the outer surface of the stent, or a combination thereof.

The ratio of the total dose in μg of the first drug to the second drug may be dependent upon the first drug and the second drug, but may range from 20:1 to 1:20.

Preferred embodiments include: methods of treatment, prevention, or amelioration of a vascular disease and/or disorder in a patent identified as having diabetes or a pre-diabetic condition and who is in need of treatment, prevention or amelioration of a vascular disease and/or disorder comprising implantation of a DES with a coating disposed over the stent including, but not limited to, everolimus, another mTOR inhibitor, or a combination thereof, and the use of a DCB, the drug of the DCB including, but not limited to, paclitaxel, an analog of paclitaxel, or a combination thereof; methods of treatment, prevention, or amelioration of a vascular disease and/or disorder in a patent identified as having diabetes or a pre-diabetic condition and who is in need of treatment, prevention or amelioration of a vascular disease and/or disorder comprising implantation of a DES with a coating disposed over the stent including, but not limited to dexamethasone, a derivative of dexamethasone, an analog of dexamethasone, methylprednisone, clobetasol, another glucoroticoid, or a combination thereof, and the use of a DCB, the drug of the DCB including, but not limited to, paclitaxel, an analog of paclitaxel, zotarolimus, everolimus, an mTOR inhibitor, or a combination thereof; methods of treatment, prevention, or amelioration of a vascular disease and/or disorder in a patent identified as having diabetes or a pre-diabetic condition and who is in need of treatment, prevention or amelioration of a vascular disease and/or disorder comprising implantation of a DES having a metal body and a coating disposed over the stent including, but not limited to, everolimus, an mTOR inhibitor, or a combination thereof, and the use of a DCB, the drug of the DCB including, but not limited to, a thiazolidinedione; methods of treatment, prevention, or amelioration of a vascular disease and/or disorder in a patent identified as having diabetes or a pre-diabetic condition and who is in need of treatment, prevention or amelioration of a vascular disease and/or disorder comprising implantation of a DES that is a bioabsorbable stent, the stent including, but not limited to, a drug in the device body of the stent, in a coating disposed over the stent, or both in the device body and in a coating disposed over the stent, the drug of the stent including, but not limited to, everolimus, an mTOR inhibitor, or a combination thereof, and the use of a DCB, the drug of the DCB including, but not limited to, a thiazolidinedione, everolimus, zotaroliums, an mTOR inhibitor, paclitaxel, an analog of paclitaxel, or a combination thereof; and methods of treatment, prevention, or amelioration of a vascular disease and/or disorder in a patent identified as having diabetes or a pre-diabetic condition and who is in need of treatment, prevention or amelioration of a vascular disease and/or disorder comprising implantation of a DES having a metal device body, a polymeric device body, or both, and a coating disposed over the stent including, but not limited to, everolimus, an mTOR inhibitor, or a combination thereof, and the use of a DCB, the drug of the DCB including, but not limited to, everolimus, sirolimus, zotarolimus, dexamethasone, another glucocorticoid, a steroidal anti-inflammatory drug, a non-steroidal anti-inflammatory drug, aspirin, naproxen, poly(aspirin), or a combination thereof.

In preferred embodiments, the stent may have a device body may from a material comprising, or consisting essentially of, a polymer, a metal, a ceramic, a glass, or a combination thereof, with one of the following coatings disposed over the stent: a polymeric coating comprising poly(lactide) and the drug Biolimus A-9; a polymeric coating comprising a combination of poly(L-lactide) and poly(lactide), and the drug everolimus; a polymeric coating comprising poly(D,L-lactide) and poly(lactide-co-glycolide), and the drug sirolimus; a polymeric coating comprising a poly(ethylene-co-vinyl acetate) and poly(n-butyl methacrylate), and the drug sirolimus; a polymeric coating comprising a methacrylate polymer, and the drug myolimus; a polymeric coating comprising a phosphorylcholine polymer, and the drug zotarolimus; a polymeric coating comprising poly(lactide) and sirolimus; a polymeric coating comprising poly(L-lactide), poly(lactide-co-glycolide), and poly(vinyl pyrrolidone), and the drug paclitaxel; a polymeric coating comprising poly(lactide-co-glycolide) and the drug sirolimus; a polymeric coating comprising a fluoropolymer, and the drug everolimus; a polymeric coating comprising a polycarbonate polymer, and the drug paclitaxel; a polymeric coating comprising poly(L-lactide), poly(lactide-co-glycolide), and poly(vinyl pyrrolidone), and the drug sirolimus; a polymeric coating comprising poly(lactide-co-glycolide), and the drugs pimecrolimus and paclitaxel; a polymeric coating comprising a styrene-iso-butylene-styrene co-polymer and paclitaxel; and a polymeric coating comprising a lipid, and the drug sirolimus. In other preferred embodiments, the stent may be a metal stent, a polymeric stent, a stent of metal and a polymer, or a stent of another material optionally in combination with a metal and/or a polymer, that elutes at least one of the following drugs: paclitaxel; pimecrolimus; an anti-CD34 drug; the combination of sirolimus and heparin; sirolimus; heparin; titanium-NO; and Biolimus A-9.

Various non-limiting embodiments of the present invention are described in the following numbered paragraphs, paragraphs (1) to (23):

(1) A method of treating, preventing, or ameliorating a vascular disease and/or disorder in a diabetic or a pre-diabetic patient, the method including, but not limited to: delivering a balloon with a coating comprising a first drug to a vascular region in a patient; deploying the drug coated balloon at the site of the vascular region to deliver the first drug; delivering a stent comprising a second drug to the vascular region of the patient; and deploying the stent comprising the second drug at the vascular region to deliver the second drug; wherein the second drug may be the same as or different from the first drug; wherein the patient is in need of treating, preventing, or ameliorating a vascular disease and/or disorder; wherein each of the first and the second drugs is independently selected from the group consisting of anti-inflammatories, thiazolidinediones, antiproliferatives, and combinations thereof; wherein the antiproliferatives are paclitaxel, docetaxel, methotrexate, azathioprine, vincristine, vinblastine, fluorouracil, doxorubicin hydrochloride, mitomycin, rapamycin (sirolimus), Biolimus A9, deforolimus, AP23572, tacrolimus, temsirolimus, pimecrolimus, novolimus, myolimus, zotarolimus (ABT-578), everolimus, 40-O-(3-hydroxypropyl)rapamycin, 40-O-[2-(2-hydroxy)ethoxy]ethyl-rapamycin, 40-O-(2-hydroxy)ethyl-rapamycin (everolimus), 40-O-tetrazolylrapamycin, 40-epi-(N1-tetrazolyl)-rapamycin, NVP-BEZ235 (an mTOR inhibitor and a PI3K inhibitor), LY294002 (available from Calbiochem), XL765 and XL147 (Exelixis/Sanofi-Aventis), GDC-0941 (Genentech/Roche), BKM120 (Novartis), BEZ235 (Novartis), AMG319 (Amgen), and CAL101 (aka GS1101) (Calistoga Pharmaceuticals/Gilead), and combinations thereof; wherein the anti-inflammatories are clobetasol, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, deflazacort, desonide, desoximetasone, dexamethasone, dexamethasone dipropionate, dexamethasone acetate, dexmethasone phosphate, momentasone, cortisone, cortisone acetate, hydrocortisone, prednisone, prednisone acetate, betamethasone, betamethasone acetate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, methylprednisolone suleptanate, morniflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, zomepirac sodium, tacrolimus, pimecrolimus, and combinations thereof; and wherein the thiazolidinediones are pioglitazone, rosiglitazone, troglitazone, netoglitazone, ciglitazone, rivoglitazone, and combinations thereof.

(2) The method described in paragraph (1), wherein the stent has a coating including, but not limited to, the second drug.

(3) The method described in any one of paragraphs (1) and (2), wherein the stent coating includes, but is not limited to, a polymer.

(4) The method described in paragraphs (1)-(3), wherein the device body of the stent includes, but is not limited to, the second drug.

(5) The method described in any one of paragraphs (1)-(4), wherein device body of the stent includes, but is not limited to, a bioabsorbable material.

(6) The method described in any one of paragraphs (1)-(5), wherein each of the first and the second drugs is an anti-inflammatory.

(7) The method described in any one of paragraphs (1)-(5), wherein the first drug is an anti-inflammatory and the second drug is an antiproliferative.

(8) The method described in any one of paragraphs (1)-(5), wherein the first drug is an antiproliferative and the second drug is an anti-inflammatory.

(9) The method described in any one of paragraphs (1)-(5), wherein the first drug is an anti-inflammatory, and the second drug is a thiazolidinedione.

(10) The method described in any one of paragraphs (1)-(5), wherein the first drug is a thiazolidinedione and the second drug is an anti-inflammatory.

(11) The method described in any one of paragraphs (1)-(5), wherein the first drug is an antiproliferative, and the second drug is a thiazolidinedione.

(12) The method described in any one of paragraphs (1)-(5), wherein the first drug is a thiazolidinedione and the second drug is an antiproliferative.

(13) The method described in any one of paragraphs (1)-(5), wherein each of the first and second drugs is a thiazolidinedione.

(14) The method described in any one of paragraphs (1)-(5), wherein each of the first and second drugs is an antiproliferative.

(15) The method described in any one of paragraphs (1)-(14), wherein the dose of the first drug is from about 1 to about 1000 ug/cm², and the dose of the second drug is from about 1 to about 1000 μg/cm² if the stent comprises a coating comprising the second drug, and from about 0.1 to about 1000 μg/cm³ if the device body of the stent comprises the second drug.

(16) The method described in paragraph (15), wherein the dose of the first drug is from about 10 to about 600 ug/cm², and the dose of the second drug is from about 10 to about 600 μg/cm² if the stent comprises a coating comprising the second drug, and from about 0.5 to about 600 μg/cm³ if the device body of the stent comprises the second drug.

(17) The method described in any one of paragraphs (1)-(16), wherein the ratio of the total dose by weight of the first drug to the second drug is in the range from about 20:1 to about 1:20.

(18) The method described in any one of paragraphs (1)-(17), wherein the stent is crimped onto the drug coated balloon and is delivered by the drug coated balloon.

(19) The method described in any one of paragraphs (1)-(17), wherein the stent is delivered and deployed prior to the delivery and deployment of the DCB, and wherein the DCB is deployed inside the stent.

(20) The method described in any one of paragraphs (1)-(17), wherein the DCB is delivered and deployed prior to the delivery and deployment of the stent.

(21) The method described in any one of paragraphs (1)-(17), wherein the DCB is delivered and deployed and the stent is implanted within a 24 hour time frame.

(22) The method described in any one of paragraphs (1)-(17), wherein the DCB is delivered and deployed and the stent is implanted within a 6 hour time frame.

(22) The method described in any one of paragraphs (1)-(17), wherein the DCB is delivered and deployed and the stent is implanted within a 6 hour time frame.

(23) The method described in any one of paragraphs (1)-(17), wherein the DCB is delivered and deployed and the stent is implanted within the same interventional procedure.

(24) The method described in any one of paragraphs (1)-(17), wherein the initiation of the expansion of the DCB begins within 30 to 90 minutes prior to the insertion of the delivery device to deliver the stent into the patient, after a dilatation balloon used to dilate the vessel is deflated, or after the deployment of the stent at the site.

(25) The method described in any one of paragraphs (1)-(24), wherein the patient is determined to have diabetes or to have a pre-diabetic condition.

While particular embodiments of the present invention have been shown and described, it will be obvious to those skilled in the art that changes and modifications can be made without departing from this invention in its broader aspects. Therefore, the claims are to encompass within their scope all such changes and modifications as fall within the true sprit and scope of this invention. Moreover, although individual aspects or features may have been presented with respect to one embodiment, a recitation of an aspect for one embodiment, or the recitation of an aspect in general, is intended to disclose its use in all embodiments in which that aspect or feature can logically be incorporated without undue experimentation.

What is claimed is:

1. A method of treating, preventing, or ameliorating a vascular disease and/or disorder in a diabetic or a pre-diabetic patient, the method comprising:
delivering a balloon with a coating comprising a first drug to a vascular region in a patient, the patient being determined to have diabetes or to have a pre-diabetic condition and patient being in need of treating, preventing, or ameliorating a vascular disease and/or disorder;
deploying the drug coated balloon at the site of the vascular region to deliver the first drug;
delivering a stent comprising a second drug, which may be the same as or different from the first drug, to the vascular region of the patient;
and
deploying the stent comprising the second drug at the vascular region to deliver the second drug;
wherein each of the first and the second drugs is independently selected from the group consisting of anti-inflammatories, thiazolidinediones, antiproliferatives, and combinations thereof;
wherein the antiproliferatives are paclitaxel, docetaxel, methotrexate, azathioprine, vincristine, vinblastine, fluorouracil, doxorubicin hydrochloride, mitomycin, rapamycin (sirolimus), Biolimus A9, deforolimus, AP23572, tacrolimus, temsirolimus, pimecrolimus, novolimus, myolimus, zotarolimus (ABT-578), everolimus, 40-O-(3-hydroxypropyl)rapamycin, 40-O-[2-(2-hydroxy)ethoxy]ethyl-rapamycin, 40-O-(2-hydroxy)ethyl-rapamycin (everolimus), 40-O-tetrazolylrapamycin, 40-epi-(N1-tetrazolyl)-rapamycin, NVP-BEZ235, XL765, XL147, GDC-0941, BKM120, BEZ235, AMG319, CAL101, GS1101, and combinations and combinations thereof;
wherein the anti-inflammatories are clobetasol, clobetasol propionate, clobetasone butyrate, dexamethasone, dexamethasone dipropionate, dexamethasone acetate, dexmethasone phosphate, momentasone, cortisone, cortisone acetate, hydrocortisone, prednisone, prednisone acetate, betamethasone, betamethasone acetate, and combinations thereof; and
wherein the thiazolidinediones are pioglitazone, rosiglitazone, troglitazone, netoglitazone, ciglitazone, rivoglitazone, and combinations thereof.

2. The method of claim 1, wherein the stent comprises a coating comprising the second drug, the device body of the stent comprises the second drug, or both.

3. The method of claim 2, wherein the stent coating comprises the second drug and a polymer.

4. The method of claim 2, wherein the device body of the stent comprises the second drug and a bioabsorbable material.

5. The method of claim 1, wherein the stent is crimped onto the drug coated balloon and is delivered by the drug coated balloon.

6. The method of claim 1, wherein the stent is delivered and deployed prior to the delivery and expansion of the drug coated balloon, and wherein the drug coated balloon is expanded inside the stent.

7. The method of claim 1, wherein the drug coated balloon is delivered and expanded prior to the delivery and deployment of the stent.

8. The method of claim 1, wherein the drug coated balloon is delivered and expanded and the stent is implanted within a 24 hour time frame.

9. The method of claim 8, wherein the drug coated balloon is delivered and expanded and the stent is implanted within a 6 hour time frame.

10. The method of claim 1, wherein the drug coated balloon is delivered and expanded and the stent is implanted within the same interventional procedure.

11. The method of claim 1, wherein the initiation of the expansion of the DCB begins within 30 to 90 minutes prior to the insertion of the delivery device to deliver the stent into the patient, after a dilatation balloon used to dilate the vessel is deflated, or after the deployment of the stent at the vascular region.

12. The method of claim 1, wherein each of the first and the second drugs is an anti-inflammatory.

13. The method of claim 1, wherein the first drug is an anti-inflammatory and the second drug is an antiproliferative.

14. The method of claim 1, wherein the first drug is an antiproliferative and the second drug is an anti-inflammatory.

15. The method of claim 1, wherein the first drug is an anti-inflammatory, and the second drug is a thiazolidinedione.

16. The method of claim 1, wherein the first drug is a thiazolidinedione and the second drug is an anti-inflammatory.

17. The method of claim 1, wherein the first drug is an antiproliferative, and the second drug is a thiazolidinedione.

18. The method of claim 1, wherein the first drug is a thiazolidinedione and the second drug is an antiproliferative.

19. The method of claim 1, wherein each of the first and second drugs is a thiazolidinedione.

20. The method of claim 1, wherein each of the first and second drugs is an antiproliferative.

21. The method of claim 2, wherein the dose of the first drug is from about 1 to about 1000 μg/cm², and the dose of the second drug is from about 1 to about 1000 μg/cm² if the stent comprises a coating comprising the second drug, and from about 0.1 to about 1000 μg/cm³ if the device body of the stent comprises the second drug.

22. The method of claim 21, wherein the dose of the first drug is from about 100 to 600 μg/cm², and the dose of the second drug is from about 10-600 μg/cm² if the stent comprises a coating comprising the second drug, and from about 0.5 to about 600 μg/cm³ if the device body of the stent comprises the second drug.
